(19) United States
(12) Patent Application Publication
(10) Pub. No.: US 2005/0277782 A1
(43) Pub. Date: Dec. 15, 2005

(54) Method for removal of acid contaminants in a process for the synthesis of dimethyl carbonate

(75) Inventors: Daniele Delledonne, Oleggio (IT); Franco Rivetti, Milano (IT)

Correspondence Address: OBLON, SPIVAK, MCCLELLAND, MAIER & NEUSTADT, P.C., 1940 DUKE STREET, ALEXANDRIA, VA, 22314, US

(73) Assignee: ENITECNOLOGIE S.p.A., San Donato Milanese-Milan (IT)

(21) Appl. No.: 10/740,574

(22) Filed: Dec. 22, 2003

(51) Int. Cl. C07C 69/96

(52) U.S. Cl. 558/280

(57) ABSTRACT

A process for treating a reaction mixture obtained by the oxidative carbonylation of an alcohol in the presence of a catalyst and hydrochloric acid includes thermally treating the condensed phase of the reaction product and at least partial evaporating the thermally treated condensed phase. The thermal treatment is carried out under conditions that decompose alkyl chloroformates present in the reaction product mixture. The thermally treated and partial evaporated mixture have a low level of chloride ion and exhibit stability over time.

### BACKGROUND OF THE INVENTION

1. Field of the Invention

The present invention relates to a method for the removal of acid contaminants from the condensed phase of a reaction mixture in a process for the synthesis of a dialkyl carbonate. The present invention further relates to an improved method for the removal of acid contaminants from the condensed phase of the reaction mixture in a process for the synthesis of dimethyl carbonate which includes subjecting the condensed phase to a thermal treatment followed by a partial evaporation carried out with a low residence time.

2. Description of the Related Art

Dimethyl carbonate (hereinafter DMC) is a widely used and extremely versatile chemical product which may be used as a solvent, an additive for fuels, an intermediate in the synthesis of other alkyl and aryl carbonates and polycarbonates, isocyanates, urethanes, ureas and several other products in the field of fine chemicals.

A method which is commonly used for the production of DMC is based on the oxidative carbonylation of methanol, according to the reaction
2CH₃OH+CO+0.5O₂→(CH₃O)₂CO+H₂O
in the presence of copper halides as catalyst and hydrochloric acid. The hydrochloric acid may be added continuously to the reaction mixture to maintain a desired molar ratio of chloride to copper.

The preparation of DMC according to this reaction is described in several patent applications. Particularly favorable recognition in industrial practice has been obtained by the process described in U.S. Pat. No. 5,210,269, U.S. Pat. No. 5,536,864 and U.S. Pat. No. 5,686,644, each of which is incorporated herein by reference in its entirety.

The above-mentioned patents describe continuous processes for the synthesis of DMC which include the removal in the vapor phase of the reaction products from the synthesis reactor. In this process, at the outlet of the reactor, there is a gaseous stream containing vapors of the H₂O, CH₃OH, and DMC, in addition to non-reacted CO and O₂, CO₂ and other organic by-products derived from secondary reactions and possibly inert gases (for example H₂ and/or N₂) present during feeding to the reactor. This mixture of gases and vapors is subjected to one or more condensation steps to separate the condensable fraction (containing for example H₂O, CH₃OH, DMC, and other minor more volatile by-products such as methylal and methyl chloride; hereafter condensed phase) from the incondensable gases. The condensed phase is then fed to the separation section which allows the recovery of the DMC and H₂O produced and the recycling of the unreacted methanol to the synthesis reactor.

The conventional process, however, has a critical aspect in that the condensed phase contains small quantities of HCl (about 100-1000 ppm by weight) and halogenated copper salts (about 1-30 mg Cu/l) derived from the catalyst and from the added HCl. The presence of chloride (and copper) ions creates considerable problems from a technical and economical point of view, as it can cause serious problems of corrosion in the plant section where product separation and purification operations are carried out with the consequent necessity of resorting to special corrosion-resistant materials and a considerable increase in costs.

The use of conventional neutralization techniques for the removal of HCl from the condensed phase, e.g. by treatment with an alkaline agent, or contact with a basic ion exchange resin is cumbersome and unsatisfactory, as it gives rise to problems relating to the precipitation and encrusting of the salts and decomposition of the DMC caused by hydrolysis in the event of an overdosing of alkaline agent. In the case of ion exchange resins, the problems arise from their low exchange capacity with the consequent necessity of using extremely high quantities of resin, effecting very frequent regenerations and disposing of high quantities of the alkaline fluids used in the regeneration.

Various processes have been proposed in the art to overcome this critical aspect.

Italian patent 1,264,936 (incorporated herein by reference in its entirety) describes the removal of HCl from the gas and vapor stream leaving the reactor by absorption with an immiscible perfluorinated organic fluid.

Patent application U.S. Pat. No. 5,527,943 (incorporated herein by reference in its entirety) describes the removal of HCl (and entrained copper) from the gas and vapor stream at the outlet of the reactor by absorption with a process fluid.

Patent application U.S. Pat. No. 5,631,395 (incorporated herein by reference in its entirety) describes the removal of HCl from a vaporized stream of the DMC process containing organic vapors and water, by absorption on an absorbing bed of alumina or activated carbon.

Particular interest has been shown in industrial practice in the process described in patent application U.S. Pat. No. 5,685,957 (incorporated herein by reference in its entirety). According to this process the removal of HCl (and copper) is achieved by partial evaporation of the condensed phase, then obtaining a vaporized stream substantially free of said contaminants and a secondary liquid stream containing the contaminants.

Not even this process, however, has proved to consistently be effective in industrial practice, especially when low residence time evaporation conditions are combined with the use of suitable evaporation equipment such as single-passage evaporators or film evaporators.

The use, on the other hand, of low residence times is preferred in many cases. In fact long residence times (on the order of hours) of the liquid at the required temperatures during the partial evaporation process of the condensed phase give rise to a significant decomposition of the DMC contained therein, with a consequent loss in yield due to the presence of water which induces hydrolysis phenomena, especially when catalyzed by high concentrations of HCl which are generated in the residual evaporation liquid. Furthermore, the use of low residence times allows the use of smaller equipment having the same throughput capacity and is preferred for high capacity production plants.

The process used to manufacture dimethylcarbonate can also be used to manufacture other dialkylcarbonates. The dimethylcarbonate or dialkylcarbonate thereby produced may be used in the preparation of polycarbonate resins produced by, for example, melt processes. The production of polycarbonate resins from dialkylcarbonate precursors offers advantages including the elimination of the necessity that a solvent be present in the formation of the polycarbonate resin and the ability to form the polycarbonate resin in processes that do not require phosgene as a precursor.

Melt processes are described in, for example, U.S. Pat. No. 3,153,008 (incorporated herein by reference in its entirety). In a typical melt process a bisphenol is reacted directly with a dialkyl or diarylcarbonate. The diarylcarbonate required for this reaction may be produced by reacting a dialkylcarbonate precursor with an aryl transfer reagent such as an arylhydroxide. Such processes are described in U.S. Pat. No. 4,182,726 (incorporated herein by reference in its entirety). The resulting diarylcarbonate may be reacted with a phenolic monomer species to provide a polycarbonate resin.

Other processes for preparing dialkylcarbonates are described in U.S. Pat. Nos. 4,218,391 and 4,318,862 (each of which is incorporated herein by reference in its entirety).

Conventional methods for preparing dialkylcarbonates may result in the formation of by-products such as water and hydrochloric acid. The presence of aqueous solutions of hydrochloric acid can be damaging to process equipment and can severely corrode materials that are not specifically corrosion resistance.

Accordingly, a process for removing acid contaminants from the condensed phase that provides a product stream substantially free of contaminant when evaporation conditions of low residence times are used is desired to reduce the problems encountered in the prior art processes.

### BRIEF DESCRIPTION OF THE DRAWINGS

FIG. 1 compares the decomposition rate of methylchloroformate with the condensed phase materials of Examples 1-3.

FIG. 2 shows the decomposition rate of methylchloroformate with the evaporate of Example 1.

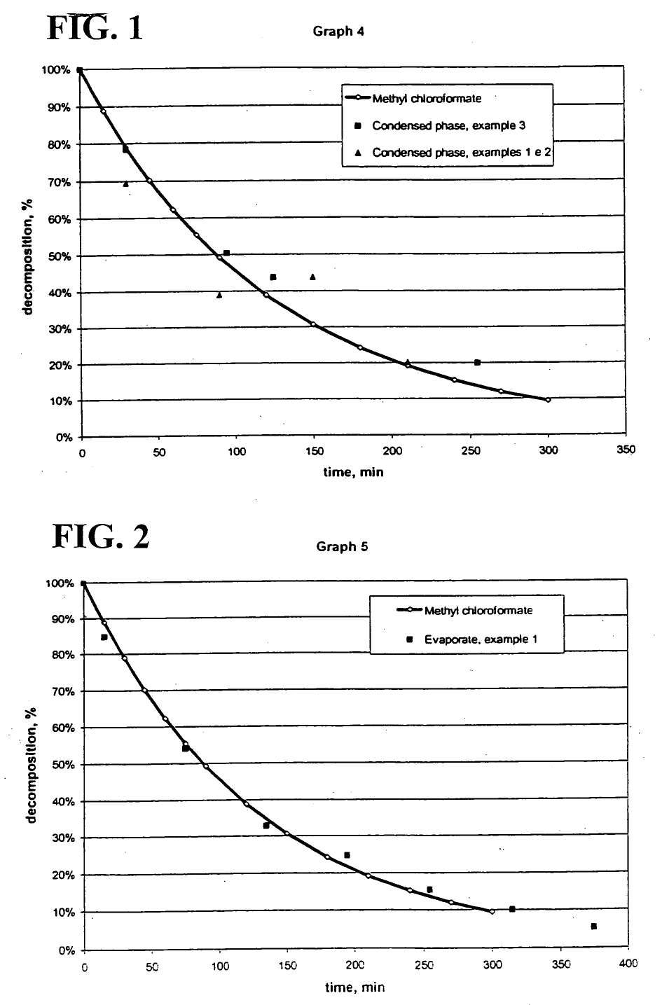

### DETAILED DESCRIPTION OF THE INVENTION

After a considerable effort, the inventors have discovered that methyl chloroformate can form during the synthesis of dimethyl carbonate as a volatile chlorinated organic by-product that, when evaporation conditions include low residence times, can pass through the HCl removal column into separation and purification steps, where it reacts slowly with methanol and/or water to form corrosive HCl.

The inventors have found that methyl chloroformate is generally present in the condensed phase at a concentration ranging from 100 to 5000 ppm by weight, normally from 300 to 3000 ppm by weight The inventors have also discovered that methyl chloroformate is moderately unstable in the condensed phase due to its hydrolysis and solvolysis reactions, as a result of the presence in the condensed phase of large quantities of methanol and water and is capable of leading to a progressive formation of HCl over a period of time, according to the reactions indicated below, as demonstrated by the increase with time of the concentration of chloride ions measured in samples of condensed phase immediately after being removed from the plant:
ClC(O)OCH₃+H₂O→CH₃OH+CO₂+HCl
ClC(O)OCH₃+CH₃OH→CH₃OC(O)OCH₃+HCl

The invention process is not limited to dimethylcarbonate. Other alkyl and aryl carbonates may also be prepared with the claimed process. Preferred processes for preparing dialkyl carbonates include reacting C₁-C₁₂ alkanols as described above for methanol.

Conventional copper catalysts such as CuCl may be used. Other catalysts including catalysts containing Fe, Ni, Co, Zn, Ru, Rh, Pd, Ag, Cd, Re, Os, Ir, Pt, Au, Hg and similar materials may also be used. Catalysts containing a more than one metal element such as CuFe may also be used. An especially preferred catalyst is CuCl where the molar ratio of the copper to chloride atoms is from 0.5 to 1.5 and a ratio of from 0.8 to 1.5 is especially preferred, even more preferred is the range 0.8 to 1.2. Other chlorides of copper or other metals mentioned above may also be suitable, such as CuCl₂. During formation of the dialkylcarbonate the chloride concentration of the catalyst may become depleted. The addition of chloride ion through, for example, the addition of HCl is preferred.

When the preferred catalyst (CuCl) is used to prepare the dialkylcarbonate a preferred catalyst concentration is from about 140 to about 180 grams of catalyst per liter of reaction mixture. The HCl concentration, is added to maintain chloride content, is preferably maintained at a feed rate of about 6×10⁻⁴ to 8×10⁻⁴ based on the weight of the HCl to the total weight of liquid added to the reactor.

Numerous experiments were carried out to determine the decomposition kinetics, at various temperatures, of any unstable species in the DMC reaction product and of methyl chloroformate in mixtures containing methanol, dimethyl carbonate, water, and in some cases hydrochloric acid.

The experiments were carried out in analogous concentrations to those present in the condensed phase obtained during the synthesis of DMC and have shown that the variations in concentration of chloride ions, over a period of time, in the condensed phase samples removed from the plant, comply with the kinetics measured for the decomposition of methyl chloroformate. On the basis of these studies, after a considerable effort, the inventors identified a process for the removal of acid contaminants from the condensed phase of the reaction effluent in a process for the synthesis of dimethyl carbonate.

One embodiment of the invention is a process for the removal of acid contaminants from the condensed phase of the reaction effluent in a process for the synthesis of dimethyl carbonate, which includes subjecting said condensed phase to:
(a) a thermal treatment, where the condensed phase is maintained within temperature and time value ranges defined by the equation:
   Log₁₀τ=−0.03*T+K
wherein τ is the residence time in hours, T the temperature in ° C. and K is a constant which has a value equal to or higher than 1.5,
(b) a partial evaporation, effected with a low residence time of the liquid in the evaporation system which, calculated with respect to the whole feeding (liquid hold-up of the apparatus/feed flow-rate) is generally equal to or less than 0.25 hours.

Operating under the above conditions the acid contaminants present in the condensed phase are removed, minimizing at the same time the hydrolytic decomposition of the DMC produced and enabling the use of lower residence time in the evaporation equipment having reduced dimensions while maintaining the same evaporation capacity.

The thermal treatment in a) stabilizes the condensed phase before evaporation, in relation to the presence of methyl chloroformate, by allowing its hydrolysis and solvolysis reactions to take place before evaporation.

Step a)

The thermal treatment includes maintaining the condensed phase within particular temperature and time value ranges, defined by the equation:
Log₁₀τ=−0.03*T+K
wherein τ is the residence time in hours, T the temperature in ° C. and K is a constant which has a value equal to or higher than 1.5, preferably 1.6 to 2.6.

In practice, in one preferred embodiment, the equation provides such values that the residence time generally ranges from 10 to 100 hrs when the temperature is 20° C. and is halved with each increase in temperature of 10° C.

The use of pairs of times and temperatures lower than those indicated by the equation makes the subsequent partial evaporation ineffective for the removal of the HCl from the vaporized stream. The use of pairs of times and temperatures higher than those indicated by the equation in the preferred embodiment above can be adopted but may cause a significant hydrolytic degradation of the DMC.

The thermal treatment is preferably carried out at temperatures ranging from 20 to 80° C., more preferably from 40 to 80° C., especially preferably from 50 to 70° C.

The pressure at which the thermal treatment is effected, is not critical and can vary within a wide range of values. It is generally convenient to adopt pressures ranging from 1 to 3 absolute bar, for example atmospheric pressure or the minimum pressure for preventing the boiling of the system at the treatment temperature.

The thermal treatment can be carried out batchwise, but is preferably effected continuously. Any type of vessel can be used for the purpose, and in particular mixing vessels or tubular-shaped vessels or combinations of the two types of vessels. The thermal treatment is preferably carried out in one (or more) tubular vessels or in a set of two or more mixing vessels arranged in series, or in a combination of one (or more) mixing vessels and at least one tubular vessel arranged in series. When the thermal treatment is effected in several vessels arranged in series, these can be maintained at different temperatures and have different residence times. In this case, in the above equation r is the overall residence time and T is the average temperature, weighted on the basis of the individual residence times.

When tubular vessels are used, these can consist of the adduction pipes themselves conveying the condensed phase to the evaporator, suitably sized and heated, traced or insulated to guarantee appropriate residence times and operating temperatures according to the invention.

The optimal residence times and temperatures are defined, within the values indicated above, in relation to the solution selected as for the type and number of the vessels. For example, the residence time values to be adopted when operating at 40° C. in some preferred embodiments, are provided below:
i. a tubular vessel: 4 h
ii. a set of 4 mixing vessels having the same volume and held at the same temperature, arranged in series: 4×2 h
iii. a combination of a mixing vessel followed by a tubular vessel with the same volume and held at the same temperature: 2×2.5 h these times being more or less halved as already mentioned for each temperature increase of 10° C.
   Step b)

The condensed phase subjected to thermal treatment in a) is fed continuously to an evaporation system, in which most of it is vaporized, generally between 80 and 99% by weight, with a residence time of the liquid calculated with respect to the whole feeding (liquid hold-up of the apparatus/feed flow-rate) equal to or less than 0.25 hours.

The evaporation is conveniently effected at atmospheric pressure or under a moderate pressure, operating for example between 1 and 3 absolute bar. Under these conditions, the evaporation temperature, in relation to the operating pressure and evaporation degree, ranges from about 65° C. to about 125° C.

Any equipment suitable for effecting partial evaporation with low residence times can be used, and in particular single-pass tube evaporators or falling film and thin film evaporators. The evaporation system can be equipped, in addition to the evaporator, with a tray distillation column or, preferably, a packed distillation column, which can be fed with the vapors coming from the evaporator for the fractionation of the vapors themselves, as known in the state of the art. The evaporation system can comprise one or a series of evaporators. For example, the liquid residue coming from the main evaporator can be sent to a second evaporator, optionally equipped with a distillation column, for the exhaustive recovery of the organic components and to concentrate, at the bottom, the residual aqueous solution of HCl, which can be conveniently recycled to the DMC synthesis reactor.

Operating under the conditions described above, methyl chloroformate or alkylchloroformate is largely decomposed before evaporation at an extent typically being higher than 90%, preferably 95%, more preferably higher than 99%; and the HCl (in addition to the possibly present copper salts) is concentrated in the liquid residue. It is most preferred to reduce the methylchloroformate (alkylchloroformate) level to less than 500 ppm by weight, more preferably less than 100 ppm and most preferably less than 30 ppm. The removal of the alkylchloroformate, including methylchloroformate, is preferably carried out in a manner so that the amount of dialkylcarbonate is not substantially effected. Preferably the decomposition of DMC is generally lower than 1% of the total DMC fed and preferably less than 0.01%, even more preferably 0%.

The HCl content in the vaporized fraction is generally less than 10 ppm by weight and preferably lower than 1 ppm. In the embodiments of the invention, before being sent to step a) or step b) described above, the condensed phase can be treated with an ion exchange resin for the removal of the copper; or the vaporized stream at the outlet of step b) before being sent to the separation and purification section of the product, can be put in contact in the vapor phase with an absorbing bed (of alumina or activated carbon) for the removal of the residual HCl; or the vaporized stream at the outlet of step b), before being sent to the separation and purification section of the product, can be put in contact, after condensation, with an ion exchange basic resin for the exhaustive removal of the residual HCl, according to what is described in more detail in U.S. Pat. No. 5,685,957.

Some illustrative but non-limiting examples are provided for a better understanding of the present invention and for its embodiment, which, however, should in no way be considered as restricting the scope of the invention itself.

In the following experimental examples, the condensed phase (the process stream coming from the synthesis reactor, after condensation and separation of the incondensable gases), was used, deriving from a plant for the production of DMC which operates according to the process described in U.S. Pat. No. 5,210,269 and U.S. Pat. No. 5,686,644.

Said liquid stream contains H₂O, CH₃OH, DMC as main products and to a much lower degree some other organic by-products, mainly methylal, as well as up to about 1000 mg/l of chloride ions. A typical weight composition is as follows:
H₂O=5% CH₃OH=55% DMC=38% Other products=2%

The chloride ions were determined on the samples of the condensed phase through potentiometric titration with a silver nitrate solution in the presence of a combined reversible electrode, sensitive to silver ions.

### EXAMPLE 1

### Comparative

A sample of the condensed phase, taken from a DMC production plant, was divided into two more or less equal aliquots. The concentration of the chloride ions as a function of the time was determined on samples of the first aliquot, kept at room temperature, obtaining the values shown in Table 1.1.

TABLE 1.1

| Time, minutes | Cl⁻, mg/l |
|---|---|
| 0 | 595 |
| 30 | 639 |
| 90 | 683 |
| 150 | 676 |
| 210 | 710 |
| 270 | 698 |
| 330 | 716 |
| 390 | 713 |

The remaining part of the first aliquot is then fed, contemporaneously and immediately, without any preliminary thermal treatment, to a continuous evaporation system, consisting of a thin film evaporator with a low residence time, equipped with a vapor condenser and collecting vessels for the evaporate and bottom residue.

The evaporation conditions are listed below:

Temperature of the heating fluid in the jacket of the thin film evaporator

| | | |
|---|---|---|
| | 86° | C. |
| Feeding | 231.3 | g |
| Evaporate | 210.7 | g |
| Residue | 20.6 | g |
| Evaporation time | 30 | min. |

The concentration of the chloride ions in relation to the time was determined on samples of the total evaporate, kept at room temperature, obtaining the values indicated in Table 1.2

TABLE 1.2

| Time, minutes | Cl⁻, mg/l |
|---|---|
| 15 | 42 |
| 30 | 65 |
| 90 | 111 |
| 150 | 143 |
| 210 | 155 |
| 270 | 169 |
| 330 | 177 |
| 390 | 184 |

The example demonstrates the intrinsic instability of the sample, the still existing instability of the evaporate and the inability, by carrying out the evaporation with a low residence time, to produce an evaporate substantially free of chloride ions, in the absence of a previous thermal treatment.

### EXAMPLE 2

The second aliquot of the sample of condensed phase collected as in example 1, was maintained at room temperature (about 25° C.) for 24 hours.

The concentration of the chloride ions was then determined on a sample of the second aliquot, obtaining a value of 738 mg/l.

The remaining part of the second aliquot was fed to the same continuous evaporation system, using the same method and operative conditions described in ex. 1.

The evaporation conditions are listed below:

Temperature of the heating fluid in the jacket of the thin

| | | |
|---|---|---|
| film evaporator | 86° | C. |
| Feeding | 302.6 | g |
| Evaporate | 273.6 | g |
| Residue | 29.0 | g |
| Evaporation time | 40 | min. |

The concentration of the chloride ions in relation to the time was determined on samples of the total evaporate, kept at room temperature, obtaining the values indicated in Table 2.1

TABLE 2.1

| Time, minutes | Cl⁻, mg/l |
|---|---|
| 10 | 3.4 |
| 30 | 4.1 |
| 90 | 4.1 |
| 150 | 3.2 |

The example demonstrates the stability of the evaporate, and the ability, by carrying out the evaporation with a low residence time, following a previous thermal treatment, under the conditions illustrated and according to the invention to produce an evaporate substantially free of chloride ions.

### EXAMPLE 3

A sample of condensed phase was collected from a plant for the production of DMC.

The concentration of chloride ions in relation to time was determined on aliquots of the sample, kept at room temperature, obtaining the values indicated in Table 3.1

TABLE 3.1

| Time, minutes | Cl⁻, mg/l |
|---|---|
| 10 | 259 |
| 30 | 299 |
| 95 | 349 |
| 125 | 361 |
| 255 | 405 |
| 355 | 419 |
| 385 | 421 |
| 405 | 421 |

At the same time, immediately after its collection, the remaining sample was placed in a flask equipped with a reflux condenser, heated in 20 minutes at 62° C. and kept at this temperature for 2 hours.

At the end of the thermal treatment, the concentration of the chloride ions in relation to time was determined on aliquots of the sample treated, kept at room temperature, obtaining the values indicated in Table 3.2

TABLE 3.2

| Time, minutes | Cl⁻, mg/l |
|---|---|
| 0 | 444 |
| 60 | 443 |
| 90 | 439 |
| 150 | 442 |

The remaining part of the treated sample was immediately fed to the thin film evaporator under the continuous evaporation conditions already described and used in examples 1 and 2, to undergo partial evaporation. 172.2 g were fed, obtaining an evaporate consisting of 148.7 g and a residue of 23.5 g.

The concentration of the chloride ions in relation to the time was determined on samples of the total evaporate, kept at room temperature, obtaining the values indicated in

TABLE 3.3

| Time, minutes | Cl⁻, mg/l |
|---|---|
| 0 | 2.0 |
| 120 | 2.5 |
| 180 | 2.9 |

The example demonstrates the stabilization of the sample and of the resulting evaporate, and the ability, by carrying out the evaporation with a low residence time, following a previous thermal treatment under the conditions illustrated and according to the invention, of producing an evaporate substantially free of chloride ions.

### EXAMPLE 4

The decomposition rate of the unstable species present a) in the condensed phase used in the examples and b) in the evaporate obtained in example 1 in the absence of treatment according to the invention, calculated from the increase with time in the concentration of the chloride ion on the basis of the data of the Tables, is compared in graphs 4 and 5 with the decomposition rate of methyl chloroformate under analogous conditions of temperature (25° C.) and composition of the mixture, measured with laboratory kinetic experiments.

The decomposition percentage (dec. %) is calculated using the formula:
dec. %=(Cl⁻_{∞}−Cl⁻_{∞})=(Cl⁻ₜ−Cl⁻₀)]/(Cl⁻¹_{∞}C⁻₀)
wherein Cl⁻₀ and Cl⁻ₜ represent the chloride ion concentration at the beginning (t=0) and at the time t, respectively, and Cl⁻_{∞} represents the maximum concentration of chloride ion reached in the already stabilized solution (t=∞).

The example demonstrates that the instability of said mixtures may be attributed to the presence of methyl chloroformate.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is note intended to be limited to the embodiments shown, but is to be accorded the widest scope consistent with the principles and features disclosed herein. Where a numerical limit or range is stated, all values and subranges therewithin are specifically included as if explicitly written out.

1. A process for the removal of one or more alkyl chloroformates from a mixture obtained by oxidative carbonylation of an alcohol in the presence of a catalyst and an acid, said process comprising
thermally treating a condensed phase of the mixture at a temperature and for a time that satisfy the following equation:
log₁₀τ=−0.03×T+K
wherein τ is the residence time in hours, T is the temperature in ° C., and K is a constant which has a value of 1.5 or greater, and
partially evaporating the thermally treated condensed phase in an evaporation apparatus having a liquid hold-up (L) by feeding the thermally treated condensed phase at a flow rate (F) into the evaporation apparatus wherein the residence time (L/F) of the thermally treated condensed phase in the evaporation apparatus is 0.25 hours or less.

2. The process as claimed in claim 1, wherein the condensed phase of the mixture comprises an alkanol, water, a dialkyl carbonate, an alkylchloroformate and hydrochloric acid.

3. The composition as claimed in claim 1, wherein the alcohol is methanol, the dialkyl carbonate is dimethyl carbonate, the alkyl chloroformate is methyl chloroformate, the acid is and hydrochloric acid and the catalyst is a copper catalyst.

4. The process as claimed in claim 1, wherein the condensed phase of the mixture comprises methanol, dimethyl carbonate, water, hydrochloric acid, and methyl chloroformate.

5. The process as claimed in claim 1, wherein the condensed phase is thermally treated at a temperature of from 20 to 80° C.

6. The process as claimed in claim 5, wherein the condensed phase is thermally treated at a temperature of from 40 to 80° C.

7. The process as claimed in claim 5, wherein the condensed phase is thermally treated at a temperature of from 50 to 70° C.

8. The process as claimed in claim 1, wherein K is from 1.6 to 2.6.

9. The process as claimed in claim 1, wherein the condensed phase is thermally treated for from 10 to 100 hours when the temperature is 20° C., and wherein the thermal treatment time is reduced by about one half for each increase in temperature of 110C.

10. The process as claimed in claim 1, wherein the thermal treatment is carried out continuously.

11. The process as claimed in claim 10, wherein the thermal treatment is carried out in one or more tubular vessels arranged in series.

12. The process as claimed in claim 11, wherein the thermal treatment is carried out at 40° C. in a tubular with a residence time of 4 h.

13. The process as claimed in claim 1, wherein the thermally treated condensed phase is partially vaporized to an extent of from 80% to 99%.

14. The process as claimed in claim 1, wherein the thermally treated condensed phase is partially vaporized at a pressure of between 1 and 3 absolute bar and from about 65° C. to about 125° C.

15. The process as claimed in claim 1, further comprising
isolating a dialkyl carbonate from the partially evaporated condensed phase.

16. The process as claimed in claim 1, wherein the chloride ion content of the partially evaporated condensed phase does not change more than 10% after standing for 150 minutes at room temperature relative to the chloride ion content of the sample measured after standing 10 minutes.

17. A dialkyl carbonate obtained by the process as claimed in claim 1.

18. Dimethyl carbonate obtained by the process as claimed in claim 1.

19. A process comprising
thermally treating a mixture for a time and at a temperature effective for reducing the alkyl chloroformate content of the mixture, then partially evaporating the thermally treated mixture, and
separating a dialkyl carbonate from the partially evaporated mixture,
wherein the chloride ion content of the partially evaporated mixture is less than 10 ppm by weight and the mixture is obtained by the oxidative carbonylation of an alkanol.

20. The process as claimed in claim 19, wherein the chloride ion content of the partially evaporated mixture is less than 1 ppm by weight.

21. The process as claimed in claim 19, wherein the mixture comprises an alkanol, water, a dialkyl carbonate, an alkylchloroformate and an acid.

22. The process as claimed in claim 19, wherein the alkanol is methanol, the dialkyl carbonate is dimethyl carbonate, and the alkyl chloroformate is methyl chloroformate.

23. The process as claimed in claim 19, wherein the mixture comprises methanol, dimethyl carbonate, water, hydrochloric acid, and methyl chloroformate.

24. The process as claimed in claim 19, wherein the mixture is thermally treated at a temperature of from 20 to 80° C.

25. The process as claimed in claim 19, wherein the mixture is thermally treated for from 10 to 100 hours hours when the temperature is 20° C., and wherein the thermal treatment time is reduced by about one half for each increase in temperature of 10° C.

26. The process as claimed in claim 19, further comprising
isolating a dialkyl carbonate from the partially evaporated condensed phase.

27. The process as claimed in claim 26, wherein the chloride ion content of the partially evaporated condensed phase does not change more than 10% after standing for 150 minutes at room temperature relative to the chloride ion content of the sample measured after standing 10 minutes.

28. A dialkyl carbonate obtained by the process as claimed in claim 19.

29. Dimethyl carbonate obtained by the process as claimed in claim 19.

30. A process for the production of an dialkylcarbonate by the oxidative carbonylation of an alcohol in the presence of a catalyst and an acid to form the dialkylcarbonate, wherein the improvement comprises
thermally treating a reaction product obtained by the oxidative carbonylation of the alkanol to decompose an alkylchloroformate.

31. A composition obtained by thermally treating a mixture comprising a dialkyl carbonate, an alkyl chloroformate, water, and alcohol, and hydrochloric acid, and then partially evaporating the mixture.
